(19) United States
(12) Patent Application Publication
(10) Pub. No.: US 2005/0085652 A1
(43) Pub. Date: Apr. 21, 2005

(54) Preparation and purification of synthetic capsaicin

(75) Inventors: Wei Chen, Naperville, IL (US); Premchandran H. Ramiya, Naperville, IL (US); Ronald M. Burch, Wilton, CT (US); Richard B. Carter, Washington Crossing, PA (US); Timothy A. Anderson, Redding, CT (US); Sharon McIlvain, Chicago Ridge, IL (US); Heping Zhang, Elmhurst, IL (US)

Correspondence Address: DAVIDSON, DAVIDSON & KAPPEL, LLC, 485 SEVENTH AVENUE, 14TH FLOOR, NEW YORK, NY, 10018, US

(73) Assignee: AlgoRx Pharmaceuticals, Inc., Cranbury, NJ (US)

(21) Appl. No.: 10/821,473

(22) Filed: Apr. 8, 2004

Related U.S. Application Data
(60) Provisional application No. 60/461,164, filed on Apr. 8, 2003.
(60) Provisional application No. 60/531,074, filed on Dec. 18, 2003.

(51) Int. Cl. C07C 231/02; A61K 31/16

(52) U.S. Cl. 554/69

(57) ABSTRACT

The present invention provides methods for synthesizing the trans isomer of capsaicin and/or capsaicin-like compounds by utilizing a process wherein the trans geometry is set from the beginning of the synthesis reaction and carried through the entire synthesis process.

This application claims the benefit of U.S. Provisional Patent Application No. 60/461,164, filed Apr. 8, 2003 and U.S. Provisional Patent Application No. 60/531,074, filed Dec. 18, 2003, the disclosures of which are hereby incorporated by reference.

### FIELD OF THE INVENTION

The present invention relates to methods of preparing and purifying synthetically prepared capsaicin.

### BACKGROUND OF THE INVENTION

Capsaicin, a pungent substance derived from the plants of the solanaceae family (hot chili peppers) has long been used as an experimental tool because of its selective action on the small diameter afferent nerve fibers C-fibers and A-delta fibers that are believed to signal pain. From studies in animals, capsaicin appears to trigger C-fiber membrane depolarization by opening cation channels permeable to calcium and sodium. Recently one of the receptors for capsaicin effects has been cloned. Capsaicin can be readily obtained by ethanol extraction of the fruit of capsicum frutescens or capsicum annum. Capsaicin is known by the chemical name N-(4-hydroxy-3-methoxybenzyl)-8-methylnon-trans-6-enamide. Capsaicin is practically insoluble in water, but freely soluble in alcohol, ether, benzene and chloroform. Therapeutically capsaicin has been used as a topical analgesic. Capsaicin is available commercially as Capsaicin USP from Steve Weiss & Co., 315 East 68^{th} Street, New York, N.Y. 10021 and can also be prepared synthetically by published methods. See Michalska et al., “Synthesis and Local Anesthetic Properties of N-substituted 3,4-Dimethoxyphenethylamine Derivatives”, Diss Pharm. Pharmacol., Vol. 24, (1972), pp. 17-25, (Chem. Abs. 77: 19271a), discloses N-pentyl and N-hexyl 3,4-dimethoxyphenylacetamides which are reduced to the respective secondary amines. Capsaicin (USP) contains not less than 110% total capsaicinoids which typically corresponds to 63% pure capsaicin. USP capsaicin is trans-capsaicin (55-60%) and also contains the precursors dihydrocapsaicin and nordihydrocapsaicin.

Although detailed mechanisms are not yet known, capsaicin mediated effects include: (i) activation of nociceptors in peripheral tissues; (ii) eventual desensitization of peripheral nociceptors to one or more stimulus modalities; (iii) cellular degeneration of sensitive A-delta and C-fiber afferents; (iv) activation of neuronal proteases; (v) blockage of axonal transport; and (vi) the decrease of the absolute number of nociceptive fibers without affecting the number of non-nociceptive fibers.

Processes for the synthesis of capsaicin and analogues thereof have been reported, for example, by Crombie et al., “Amides of Vegetable Origin Part VI Synthesis of Capsaicin,” Journal of the Chemical Society, pp. 1025-1027 (1955) describes an unambiguous synthesis of capsaicin, N-(4-hydroxy-3-methoxybenzyl)-8-methylnon-trans-6-enamide, the active principle in red pepper. U.S. Pat. No. 4,493,848 issued to LaHann et al. on Jan. 15, 1985, describes N-[(substituted phenyl)methyl]-cis-monosaturated alkenamide compositions and methods of synthesizing the same for parenteral, oral and topical administration. U.S. Pat. No. 5,094,782 issued to Chen et al. on Mar. 10, 1992, describes synthesis of nonanoyl vanillylamide succinate from synthetic capsaicin (nonanoyl vanillylamide) and succininc anhydride.

In addition, other references report utilizing a Wittig reaction as the key step for the introduction of the double bond. However, the Wittig reaction always favors a cis alkene product. Thus additional steps, including fractional recrystallization, are required to isomerize and separate the cis product to the trans product.

Alternative methods for the synthesis of capsaicin utilize the Claisen ester rearrangement as a means to selectively form the trans isomer of the olefin (alkene) double bond. Although superior to a Wittig reaction since a cis product is not possible, the use of the Claisen ester rearrangement produces an initial product, in which the carbon chain is too short. Thus, the chain must be lengthened after the Claisen step, prior to coupling with the benzyl amine to form the final product.

It would be advantageous to provide a method for the synthesis of the trans isomer of capsaicin so as to provide for a total synthesis of the trans isomer without the additional steps required for the synthesis of capsaicin described in the prior art.

Further, it would be advantageous to provide a method for purifying synthetic capsaicin prepared by the methods described herein.

The present invention is directed in part to the fact that the trans geometry is set from the beginning of the synthesis reaction and carried through a straightforward four-step process. Moreover, the present invention is directed in part to the fact that synthetically prepared trans capsaicin can be purified with 99.0% or greater purity.

### OBJECTS AND SUMMARY OF THE INVENTION

It is an object of the present invention to provide a method for the total synthesis of the trans isomer of capsaicin or capsaicin-like compounds.

It is another object of the present invention to provide a method for purifying the capsaicin or capsaicin-like compounds of the present invention.

In accordance with the above objects and others, in certain preferred embodiments of the present invention, there is provided a method for synthesizing the trans isomer of capsaicin from a four step process.

In certain other embodiments, there is provided a method for preparing trans-capsaicin, comprising a) alkylating 3-methyl butyne with halovaleric acid and/or ω-haloalkanic acid to obtain 8-methyl-6-nonynoic acid and/or alkynoic acid analogues thereof having the following formula (wherein n=4-8): 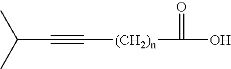
b) reducing said 8-methyl-6-nonynoic acid to obtain trans-8-methyl-nonenoic acid; c) activating the 8-methyl-nonenoic acid to obtain an acid chloride; and d) acylating 4-hydroxy-3-methoxybenzylamine hydrochloride with the acid chloride to obtain transcapsaicin.

In certain other embodiments, there is provided a method for preparing trans-capsaicin wherein step a) comprises the steps of: i) mixing anhydrous tetrahydrofuran (THF) with hexamethylphosphoramide (HMPA) and cooling the mixture to about −78° C. to about −75° C.; ii) adding to the mixture of step i) 3-methyl butyne followed by a dropwise addition of a base at a temperature from about −78° C. to about −65° C. to obtain a second mixture; iii) warming the second mixture up to about −30° C. and stirring (preferably for about 30 minutes); and iv) adding dropwise a solution of a halovaleric acid in anhydrous tetrahydrofuran at a temperature of about −30° C., the halovaleric acid added in a sufficient amount to convert said 3-methyl butyne to 8-methyl-6-nonynoic acid, then gradually warming to room temperature and stirring (preferably overnight) to obtain a reaction mixture.

In certain embodiments, there is provided a method for obtaining a crude step a) intermediate product further comprising the steps of: i) adding hydrochloric acid (HCl) to a reaction mixture (preferably 3M HCL) and extracting the reaction mixture with ethyl acetate; and ii) washing the extracted reaction mixture with brine to yield a crude product.

In another embodiment of the invention there is provided a method of purifying the crude step a) intermediate product comprising the steps of: i) purifying the crude product by column chromatography using silica gel and eluting with a mixture of ethyl acetate/hexane; and ii) removing solvents under vacuum to provide a step a) intermediate product.

In certain other embodiments, there is provided a method for preparing trans-capsaicin wherein step b) comprises the steps of: i) dissolving said 8-methyl-6-nonynoic acid in a mixture of anhydrous tetrahydrofuran and tertiary-butyl alcohol (t-BuOH) to obtain a solution and cooling the solution to about −55° C. to about −40° C.; ii) condensing ammonia (NH₃) to the solution to a temperature of about −50° C. to about −40° C.; iii) adding sodium drips piece-wise at a temperature from about −45° C. to about −30° C. and stirring for a sufficient amount of time to dissolve the sodium (preferably from about 30 minutes to about 2 hours), and iv) adding ammonium chloride (NH₄Cl), warming to room temperature an allowing the NH₃ to evaporate overnight to obtain a reaction mixture.

In certain other embodiments, there is provided a method for preparing trans-capsaicin wherein step iii) of the step b) reaction further comprises adding piece-wise lithium at a temperature from about −65° C. to about −45° C. and stirring for a sufficient amount of time to dissolve the lithium (preferably from about 30 minutes to about 2 hours).

In certain other embodiments, there is provided a method for preparing trans-capsaicin wherein step b) further comprises the steps of: i) stirring said reaction mixture overnight to evaporate ammonia; ii) adding additional anhydrous tetrahydrofuran and ammonium chloride, stirring said mixture for a sufficient time to neutralize excess lithium (preferably 30 minutes); iii) adding ice-water portionwise; iv) extracting said mixture with ethyl acetate, washing with brine and drying over anhydrous sodium sulfate; and v) filtering and removing solvents under vacuum to produce a step b) intermediate product.

In certain embodiments, there is provided a method for obtaining a crude step b) intermediate product further comprising the steps of: i) adding water to a reaction mixture; ii) acidifying the reaction mixture with HCl (preferably 6N HCL) to a pH of about 2 to about 3; iii) extracting the reaction mixture with ethyl acetate, washing with brine and drying over anhydrous sodium sulfate (Na₂SO₄); and iv) filtering and removing solvents under vacuum to obtain a crude step b) intermediate product.

In another embodiment of the invention there is provided a method of purifying the crude step b) intermediate product comprising the steps of: i) purifying the product by flash column chromatography using silica gel and eluting with a mixture of ethyl acetate/hexane to obtain a step b) intermediate product.

In certain other embodiments, there is provided a method for preparing trans-capsaicin wherein step c) comprises the steps of: i) adding dropwise a thionyl halide to the 8-methyl-6-nonenoic acid at room temperature to form a solution; ii) heating the solution at about 50° C. to about 75° C. for a sufficient period of time to convert said 8-methyl-6-nonenoic acid to an acid halide (preferably about 1 hour); and iii) removing excess thionyl halide under vacuum at about 40° C. to about 45° C. to obtain a step c) intermediate product.

In certain other embodiments, there is provided a method for preparing trans-capsaicin wherein step d) comprises the steps of: i) mixing 4-hydroxy-3-methoxy benzylamine hydrochloride and dimethylformamide (DMF); ii) adding portion-wise at room temperature to the mixture of step i) aqueous sodium hydroxide (preferably 5N NaOH) and stirring (preferably for about 30 minutes); iii) adding acid halide in anhydrous ether dropwise at a temperature of about 0° C. to about 10° C. for a sufficient period of time to convert the acid halide to an amide (preferably about 20 minutes to about 1 hour); and, thereafter, iv) gradually warming the mixture to room temperature and stirring (preferably overnight).

In certain embodiments, there is provided a method for obtaining a crude trans-capsaicin product of step d) further comprising the steps of: i) adding water to the mixture and extracting the mixture with ethyl acetate to obtain an ethyl acetate extract; ii) washing said extract with HCl (preferably 1N HCL) and, thereafter, washing with sodium bicarbonate (NaHCO₃); iii) washing the solution with brine and drying over anhydrous sodium sulfate (Na₂SO₄); and iv) filtering and removing solvents under vacuum to obtain a crude product.

In another embodiment of the invention there is provided a method of purifying the crude trans-capsaicin step d) product comprising purifying the crude product by column chromatography using silica gel and eluting with a mixture of ethyl acetate/hexane to obtain a crude trans-capsaicin product.

In certain other embodiments, there is provided an additional method of purifying the trans-capsaicin product comprising the steps of: i) dissolving the crude trans-capsaicin product in a mixture of ether/hexane and heating the mixture to about 40° C. to about 45° C.; ii) cooling the mixture to room temperature while stirring for about 2 hours; and iii) filtering the mixture to provide a purified trans-capsaicin product.

In certain other embodiments, there is provided a method of purifying capsaicin via High Performance Liquid Chromatography (HPLC) using a semi-preparative HPLC.

In certain embodiments the present invention is further directed to a method of purifying a crude trans-capsaicin product of the present invention or further purifying a previously purified trans-capsaicin product of the present invention. Preferably the purification provides for an ultra-purified trans-capsaicin product having a purity of about 97% or greater, preferably about 98% or greater, more preferably about 99% or greater. Such purification is also referred to herein as a “semi-prep purification” or semi-preparative purification of capsaicin. The semi-prep purification of capsaicin in accordance with the present invention is preformed using a semi-preparative HPLC. In certain preferred embodiments the capsaicin is previously purified prior to a further purification of the capsaicin via the semi-preparative HPLC.

In certain embodiments, the present invention is further directed to an ultra-purified capsaicin product prepared in accordance with the present invention, wherein the capsaicin product has a purity of greater than about 97%, preferably greater than about 98%, more preferably greater than about 99% capsaicin.

In certain other embodiments, there is provided a capsaicin composition for relieving pain at a site in a human or animal in need thereof consisting essentially of trans capsaicin. Preferably the capsaicin composition comprises the ultra-purified capsaicin of the present invention and a suitable vehicle for administration (e.g., via injection or infiltration).

In certain other embodiments, there is provided a composition comprising trans-capsaicin or trans-capsaicin like compounds for the treatment of various conditions associated with pain, for example, nociceptive pain (pain transmitted across intact neuronal pathways), neuropathic pain (pain caused by damage to neural structures), pain from nerve injury (neuromas and neuromas in continuity), pain from neuralgia (pain originating from disease and/or inflammation of nerves), pain from myalgias (pain originating from disease and/or inflammation of muscle), pain associated with painful trigger points, pain from tumors in soft tissues, pain associated with neurotransmitter-dysregulation syndromes (disruptions in quantity/quality of neurotransmitter molecules associated with signal transmission in normal nerves) and pain associated with orthopedic disorders such as conditions of the foot, knee, hip, spine, shoulders, elbow, hand, head and neck. Preferably the trans-capsaicin or trans-capsaicin like compounds are ultra-purified.

In order that the invention described herein may be more fully understood, the following definitions are provided:

The term “trans capsaicin” as used herein encompasses both the trans isomer of capsaicin and the trans isomer of all capsaicin-like compounds prepared by the methods of the present invention.

The term “capsaicin receptor” as used herein encompasses the vanilloid receptor subtype-1 (VR1) described in detail herein, but is not meant to be limited to VR1, and particularly may be generically used to refer to the receptor subtypes VR1 and VR2.

### DETAILED DESCRIPTION OF THE INVENTION

The methods disclosed herein can be useful for the synthesis of trans capsaicin or trans capsaicin-like compounds.

In certain other embodiments of the present invention, trans capsaicin is synthesized by the following chemical reaction:

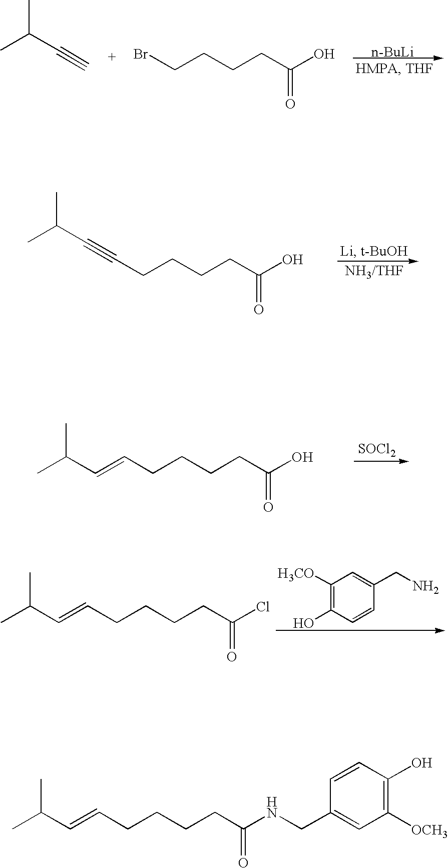

In a first step in the synthesis of trans capsaicin, a first intermediate is preferably synthesized by an alkylation reaction. In one preferred embodiment the first intermediate synthesized is preferably 8-methyl-6-nonynoic acid. The 8-methyl-6-nonynoic acid is preferably synthesized by alkylation of 3-methyl-butyne with a halovaleric acid such as bromovaleric acid, chlorovaleric acid, fluorovaleric acid, iodovaleric acid astatinovaleric acid, 1-mesyloxyvaleric acid and 1-tosyloxyvaleric acid.

The alkylation reaction is preferably driven by the addition of solvents such as hexamethylphosphoramide and tetrahydrofuran in the presence of a suitable base, e.g., n-butyllithium (n-BuLi). In certain other embodiments of the present invention, alternative bases such as secondary butyllithium (sec-BuLi), tertiary butyllithium (t-BuLi), lithium di(isopropyl) amide (LDA), sodium hydride (NaH), sodium amide (NaNH₂), lithium amide (LiNH₂), methyl lithium (MeLi), methyl magnesium bromide (MeMgBr), ethyl magnesium bromide (EtMgBr), alkyl or aryl magnesium halides and mixtures thereof can be used instead of butyllithium during the alkylation step.

In certain other embodiments, the hexamethylphosphoramide can be replaced by 1,2-dimethyl-3,4,5,6-tetrahydro-(1H) pyrimidinone.

In certain other embodiments, additional solvents such as ether may preferably be used during the alkylation reaction step.

The crude product from step one synthesis is preferably purified by column chromatography.

In certain other embodiments, the step 1 intermediate can be purified by acid-base extractions.

In certain other embodiments, the step 1 intermediate can be purified by vacuum distillation or fractional vacuum distillation or low temperature crystallization.

In a second step in the synthesis of trans capsaicin, a second intermediate is preferably synthesized by reduction of the first intermediate. In one preferred embodiment the second intermediate synthesized is preferably 8-methyl-6-nonenoic acid.

The reduction reaction is preferably driven by lithium, t-BuOH, NH₃/THF. However, in certain other embodiments, metal hydrides such as diisobutylaluminum hydride (DIBAL-H), sodium in liquid ammonia, lithium with lower alkyl amines can preferably be used in the reduction step to give the desired product. The percent yield of the desired product will vary depending on the agent chosen to complete the reduction step.

In certain other embodiments, the t-BuOH can be replaced by other alkyl alcohols such as secondary butyl alcohol (sec-BuOH), ethyl alcohol (EtOH).

In a third step in the synthesis of trans capsaicin, a third intermediate is preferably synthesized by activation of the second intermediate with a thionyl halide, e.g., thionyl chloride. In one preferred embodiment the third intermediate synthesized is preferably a acid halide, e.g., acid chloride, having the following formula:

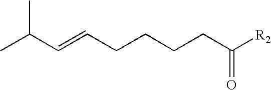

Wherein R₂ is selected from the group consisting of chlorine, bromine, imidazolides, carbodiimide and other cleaving groups such as mixed esters.

In certain other preferred embodiments, oxalyl chloride, phosphorous pentachloride, phosphorous trichloride, and sulfuryl chloride may preferably be used instead of a thionyl halide.

In certain other embodiments, the activation of carboxylic acid can be achieved by formation of mixed esters with isobutyl chloroformate, imidazolides, and carbodiimide.

In certain other embodiments, activation can be achieved with 1,3-dicyclohexylcarbodiimide (DCC), 1-ethyl-3-(3′-dimethylaminopropyl)carbodiimide hydrochloride (EDCI), combination of DCC and 1-hydroxybenzotriazole (HOBt) or 1-hydroxy-7-azabenzotriazole (HOAt), a combination of EDCI and HOAt or HOBt, or carbonyldiimidazole (CDI), thiocarbonylimidazole instead of thionyl halide.

In a fourth step in the synthesis of trans capsaicin, the final trans capsaicin product is synthesized by acylation of a benzylamine derivative with the acid halide. In one preferred embodiment, the benzylamine derivative is 4-hydroxy-3-methoxybenzylamine hydrochloride.

In certain embodiments the 4-hydroxy-3-methoxy benzylamine HCl salt can be replaced by 4-hydroxy-3-methoxy benzylamine to react with the acid chloride.

In certain embodiments, the acylation reaction with the acid halide is driven by aqueous sodium hydroxide (NaOH) in dimethylformamide (DMF) and ethyl ether (Et₂O).

In other preferred embodiments, alternative bases such as potassium hydroxide (KOH), lithium hydroxide (LiOH), sodium carbonate, potassium carbonate, alkyl amines such as triethylamine, Hunig's base, 4-dimethylaminopyridine (DMAP), and pyridine can preferably be used instead of sodium hydroxide.

In certain other embodiments, alternative solvents such as tetrahydrofuran, 1,2-dimethoxyethane (DME), acetonitrile, methyl ethyl ketone (MEK), dichloromethane and chloroform can preferably be used in place of dimethylformamide/ether.

In certain embodiments, in a fifth and final step the final (crude) trans-capsaicin product is purified by recrystallization. Preferably, recrystallization includes purifying the trans-capsaicin product comprising the steps of: (i) dissolving the crude trans-capsaicin product in a mixture of ether/hexane and heating the mixture to about 40° C. to about 45° C.; ii) cooling the mixture to room temperature while stirring for about 2 hours; and iii) filtering the mixture to provide a purified trans-capsaicin product. Alternatively, the final (crude) trans capsaicin product may be purified by column chromatography using silica gel and eluting with e.g., a mixture of ethyl acetate/hexane to obtain a purified trans-capsaicin product.

Additionally, or alternatively to the above-mentioned fifth and final step, the final trans-capsaicin product is purified via a semi-preparative HPLC to provide an ultra-purified trans-capsaicin having a purity of about 97% or greater, preferably about 98% or greater, more preferably about 99% or greater.

In certain embodiments, the semi-preparative HPLC system is for example, an adsorption chromatography system, an ion-exchange chromatography system, a size exclusion chromatography, or the like. Preferably the HPLC system is an adsorption chromatography system such as a reverse phase chromatography system.

In certain embodiments, the final purification step of the present invention includes performing the semi-preparative HPLC through the use of an isocratic elution (e.g., an isocratic mobile phase) or gradient elution (e.g., a gradient mobile phase). In isocratic elution, the compounds (e.g., capsaicin and the impurities) are eluted using a mobile phase having a constant composition. The compounds migrate through the column at onset, with each compound migrating at a different rate, resulting in separation of the compounds. In gradient elution, the compounds may be eluted as the composition of the mobile phase changes, e.g., by increasing the concentration and/or strength of the organic solvent.

Mobile phases for use in the present invention typically include for example, acetonitrile, dioxane, ethanol, isopropanol, hexane, EtOAc, methanol, tetrahydrofuran, water, combinations thereof, and the like. Most preferably the mobile phase comprises methanol.

Semi-preparative HPLC columns for use in accordance with the present invention include for example and without limitation, Symmetry C18, Cogent HPS C18, Zorbax SB-C18 StableBond, Hichrom C18, Genesis 300 C18, OmniSphere C18, HxSil C18, and the like.

In certain preferred embodiments, the trans-capsaicin prepared by the methods of the present invention has a purity of about 97% or greater, about 98% or greater, or 99% or greater capsaicin.

In certain embodiments, the present invention is further directed to a trans-capsaicin compound having a purity of about 97% or greater, about 98% or greater, or about 99% or greater capsaicin. Such capsaicin is also referred to herein as ultra-purified capsaicin.

In certain embodiments, the present invention is further directed to an ultra-purified capsaicin trans-capsaicin compound having an impurity level of about 3% or less, about 2% or less, or about 1% or less.

The trans capsaicin prepared by the methods of the present invention is preferably suitable for the preparation of an injectable or infiltratable composition which can be administered to a discrete site in a human or animal for the treatment of pain.

As used herein, the terms “trans capsaicin” and “trans capsaicin-like compounds” include trans isomers of capsaicin and capsaicin-like compounds that act at the same pharmacologic sites, e.g., vanilloid receptor subtype-1, as capsaicin, unless otherwise specified. Capsaicin-like compounds with similar physiological properties, i.e., triggering C fiber membrane depolarization by opening of cation channels permeable to calcium and sodium, are known. For example, U.S. Pat. No. 4,812,446 issued to Brand (Procter & Gamble Co.) on Mar. 14, 1989 describes other capsaicin-like compounds and methods for their preparation. U.S. Pat. No. 4,424,205 issued to LaHann on Jan. 3, 1984 cites capsaicin-like analogues. Ton et al., Brit. J. Pharm. 10:175-182 (1955) discusses the pharmacological actions of capsaicin and its analogues.

Where a trans capsaicin-like compound is synthesized by the methods of the present invention, the trans capsaicin-like compound will preferably provide similar physiological properties to trans capsaicin as are known in the art.

Suitable trans capsaicin-like compounds preferably include, but are not limited to homocapsaicin, eugenol, curcumin, anandamide, piperine, piperyline, piperettine, piperolein A, piperolein B, piperanine and any combinations or mixtures thereof.

The trans capsaicin and trans capsaicin-like compounds synthesized by the methods of the present invention are especially useful for treating disorders or pain that can be alleviated through activation of the vanilloid receptors as trans isomers are recognized mediators of the VR-1 mechanism. The synthetic method of the invention results in a capsaicin product which consists essentially of trans-capsaicin. The capsaicin product prepared in accordance with the invention contains less than 1% cis-capsaicin.

A vanilloid moiety constitutes an essential structural component of trans capsaicin and trans capsaicin-like compounds, therefore, the proposed site of action of these trans compounds has been more generally referred to as the vanilloid receptor (Szallasi 1994 Gen. Pharmac. 25:223-243). In contrast, while the cis-isomer of capsaicin has activity via a number of mechanisms, VR-1 is not considered to comprise a major effect of that agent.

VR-1 is a Ca²⁺ permeable non-selective cation channel that is activated by vanilloids, e.g., capsaicin and resiniferatoxin. The human vanilloid receptor when expressed in mammalian cells is activated by capsaicin, temperatures greater than 42° C. and by pH less than 5.5. The activation by all these effectors can be blocked substantially or completely by the action of the capsaicin antagonist capsazepine.

VR-1 is found along the entire length of primary sensory neurons with somata I dorsal-root and trigeminal ganglia. These neurons are of small to medium diameter and give rise to unmyelinated C-fibers. VR-1 positive neurons with C-fibers can be divided into two subdivisions: peptidergic and nonpeptidergic. Among the neuropeptides found in vanilloid-sensitive neurons, substance P and CGRP are the best characterized. Non-peptidergic vanilloid sensitive neurons characteristically possess the P2X₃ purinoceptor and cross-desensitization between purinoceptors and vanilloid receptors. A subset of nodose ganglion neurons also contain VR-1. VR-1 is believed to function as a shared receptor for various noxious stimuli, including heat, acids and some plant toxins. In addition, during inflammation, endogenous substances released from activated immune cells might also target VR-1, whose activation, apart from nociception, also leads to CGRP-mediated local vasodilation. In the gastrointestinal tract, this mechanism is believed to have a central role in mucousal protection. By contrast, the role of VR-1 in the central terminals of primary neurons, i.e., the dorsal horn of the spinal cord and the endogenous activators of this receptor are unknown.

Vanilloids such as trans-capsaicin, have a biphasic action on sensitive peripheral nerves, an initial excitatory phase (manifested as pain and/or neurogenic inflammation) followed by a lasting refractory state, traditionally known as desensitization (Szallasi 2000 Trends Neurosci. 25:491-497).

The trans capsaicin compositions of the present invention can be used for treating various conditions associated with pain by providing pain relief at a specific site. Examples of conditions to be treated include, but are not limited to, The compositions and methods of the present invention can be used for treating various conditions associated with pain by providing pain relief at a specific site. Examples of conditions to be treated include, but are not limited to, nociceptive pain (pain transmitted across intact neuronal pathways), neuropathic pain (pain caused by damage to neural structures), pain from nerve injury (neuromas and neuromas in continuity), pain from neuralgia (pain originating from disease and/or inflammation of nerves), pain from myalgias (pain originating from disease and/or inflammation of muscle), pain associated with painful trigger points, pain from tumors in soft tissues, pain associated with neurotransmitter-dysregulation syndromes (disruptions in quantity/quality of neurotransmitter molecules associated with signal transmission in normal nerves) and pain associated with orthopedic disorders such as conditions of the foot, knee, hip, spine, shoulders, elbow, hand, head and neck.

In certain embodiments the present invention is further directed to a pharmaceutical composition comprising the capsaicin prepared in accordance with the present invention (e.g., the ultra purified capsaicin). Preferably the composition comprises the capsaicin prepared in accordance with the present invention, (e.g., the ultra purified capsaicin) and a vehicle suitable for administration to a human or an animal. Preferably the capsaicin is incorporated into the vehicle. More preferably the vehicle is suitable for infiltration or injection administration to a human or animal.

In certain embodiments, the capsaicin is dissolved in a vehicle such as oils, propyleneglycol or other solvents commonly used to prepare injectable or infiltratable solutions. Suitable pharmaceutically acceptable vehicles preferably include aqueous vehicles, nonaqueous vehicles, antimicrobial agents, isotonic agents, buffers, antioxidants, suspending and dispersing agents, emulsifying agents, sequestering or chelating agents and any combinations or mixtures thereof. Examples of aqueous vehicles preferably include Sodium Chloride Injection, Bacteriostatic Sodium Chloride Injection, Ringers Injection, Isotonic Dextrose Injection, Sterile Water Injection, Bacteriostatic Sterile Water Injection, Dextrose Lactated Ringers Injection and any combinations or mixtures thereof. Nonaqueous parenteral vehicles preferably include fixed oils of vegetable origin, cottonseed oil, corn oil, sesame oil, peanut oil and any combinations or mixtures thereof. Additional pharmaceutically acceptable vehicles also preferably include ethyl alcohol, polyethylene glycol, glycerin and propylene glycol for water miscible vehicles and sodium hydroxide, hydrochloric acid, citric acid or lactic acid for pH adjustment and any combinations or mixtures thereof. Any combinations of the aforementioned vehicles may be used. A preferred vehicle for use in accordance with the present invention comprises about 20% PEG 300, about 10 mM histidine and about 5% sucrose in water for injection.

Alternatively, or additionally, one or more of the following agents may also be included in the compositions of the present invention:

Antimicrobial agents for use in the composition include bacteriostatic or fungistatic concentrations preferably include phenols, cresols, mercurials, benzyl alcohol, chlorobutanol, ethyl and propyl p-hydroxybenzoic acid esters, thimerosal, benzalkonium chloride benzethonium chloride and mixtures thereof;

Isotonic agents for use in the present composition preferably include sodium chloride, dextrose and any combinations or mixtures thereof;

Buffers for use in the compositions of the present invention preferably include acetate, phosphate, citrate and any combinations or mixtures thereof;

Antioxidants for use in the compositions of the present invention preferably include ascorbic acid, sodium bisulfate and any combinations or mixtures thereof;

Suspending and dispersing agents for use in the compositions of the present invention preferably include sodium carboxymethylcelluose, hydroxypropyl methylcellulose, polyvinylpyrrolidone and any combinations or mixtures thereof;

Emulsifying agents for use in the compositions of the present invention preferably include Polysorbate 80 (Tween 80).

Sequestering or chelating agents of metal ions for use in accordance with the present invention preferably include ethylenediaminetetraacetic acid.

Preferably, when the single dose of capsaicin is administered separately from or without local anesthetic, the dose of capsaicin can preferably be combined with a pharmaceutically acceptable vehicle for injection or infiltration.

Depending on the pharmaceutically acceptable vehicle chosen, in certain embodiments, the single dose of capsaicin can be administered as an aqueous solution or suspension for injection or infiltration.

### DETAILED DESCRIPTION OF THE PREFERRED EMBODIMENTS

The following Examples illustrate various aspects of the present invention. They are not to be construed to limit the claims in any manner whatsoever.

### Step 1: Alkylation of 3-methyl-butyne with Bromovaleric Acid

The first step in the synthesis of trans capsaicin involves the synthesis of a first intermediate, e.g., (8-methyl-6-nonynoic acid) by alkylation of 3-methyl-butyne with a halovaleric acid, e.g., bromovaleric acid. A first intermediate composition was successfully synthesized in the laboratory by Examples I(a)-(e) as follows:

### EXAMPLE I(a)

8-methyl-6-nonynoic acid was prepared under two separate reactions (reaction 1 and reaction 2) by adding anhydrous tetrahydrofuran (15 ml) and hexamethylphosphoramide (3.8 ml) to a 50 ml 3-necked RB flask equipped with a thermometer, a magnetic stirrer and nitrogen in/outlet. The mixture was cooled to about −78° C. to about −75° C. Methyl-butyne (1 g) was then added followed by dropwise addition of 2.5M n-BuLi (Rxn 1=1 equiv. and Rxn 2=2 equiv.) at a temperature from about −78° C. to about −65° C. The mixtures in both reactions were gradually warmed up to about −30° C. and stirred at this temperature for about 30 minutes. A solution of 5-bromovaleric acid (1.33 g in 3 ml of THF) was added dropwise at about −30° C. for about 10 to about 15 minutes. The mixtures of reaction 1 and reaction 2 were then gradually warmed to room temperature and stirred overnight.

The reaction mixtures (rxn 1 and 2) were then warmed up. Approximately 50 ml of 3M HCl was added to both the solution of reaction 1 and the solution of reaction 2. The solutions were then extracted with ethyl acetate (2×100 ml) and washed with brine. The extraction yielded about 1.1 g of crude product from reaction 1 and about 0.8 g of crude product from reaction 2.

The crude product from reaction 1 was purified by column chromatography using about 50 g of silica gel and eluted with a 2:1 mixture of hexane/ethyl acetate. The collections were combined. Solvents were removed under vacuum to give a step 1 intermediate product (8-methyl-6-nonynoic acid). The intermediate product produced was a light yellow oil. The amount of intermediate produced weighed about 0.55 g (yield=46%).

### EXAMPLE I(b)

8-methyl-6-nonynoic acid was prepared by adding hexamethylphosphoramide (30 ml) and anhydrous tetrahydrofuran (120 ml) under nitrogen to a 500 ml 3-necked BR flask equipped with a mechanical stirrer, an additional funnel and a thermometer. The mixture was cooled to about −78° C. to about −70° C. 3-methyl-butyne 7.9 gm (11.8 ml) was added at −75° C. followed by dropwise addition of 2.5M n-BuLi (46 ml) for about 20 minutes. The mixture was then warmed to −30° C. while stirring for about 45 minutes. A solution of bromovaleric acid (10.4 g in approximately 20 ml anhydrous THF) was added dropwise at about −30° C. to about 25° C. for about 20 minutes. The mixture was then gradually warmed to room temperature and stirred overnight. A TLC of the reaction mixture showed no starting material present.

3M HCl (100 ml) and water (100 ml) were added to the mixture. The temperature was increased to about 3° C. The mixture was then extracted with ethyl acetate (3×200 ml). The extractions were combined and washed with brine. The organic layer was dried over anhydrous sodium sulfate, filtered and the solvents were removed under vacuum at approximately 35° C. to give a crude step 1 end product that was a light orange/yellow oil. The crude end product was purified by column chromatography using silica gel (approx. 250 g) and eluted with a 1:2 mixture of ethyl acetate/hexane. The collections were combined and solvents were removed under vacuum to produce the step 2 intermediate as a light yellow oil. The weight was about 6.7 g (71% yield).

### EXAMPLE I(c)

Hexamethylphosphoramide (60 ml) and anhydrous tetrahydrofuran (240 ml; containing 250 ppm BHT inhibitor) were added to a 1L 3-necked RB flask equipped with a mechanical stirrer, an additional funnel and a thermometer under nitrogen in/outlet. The solution was cooled to −70° C. 3-methyl-butyne was added 15.7 gm (23.6 ml) to the solution. Next, 2.5M n-BuLi (92 ml) was added dropwise at a temperature less than about −50° C. for 25 minutes. The solution was gradually warmed to −30° C. and stirred for 50 minutes. A solution of 5-bromovaleric acid (20.8 g) in anhydrous tetrahydrofuran (40 ml) was added dropwise at a temperature of about −30° C. to about −25° C. for about 30 minutes. The solution was gradually warmed to room temperature and stirred overnight. A TLC of the reaction mixture indicated a cleaner reaction.

The reaction mixture was cooled to 5° C. to 10° C. 3M HCl (100 ml) and water (150 ml) were added to the solution. The resulting mixture was extracted with ethyl acetate (3×200 ml). TLC of the ethyl acetate extraction showed that two ethyl acetate extractions were enough. The two ethyl acetate extractions were combined, washed with brine (350 ml), dried over anhydrous Na₂SO₄ and filtered. The solvents were removed under vacuum to give about 25.8 g of intermediate product.

The crude intermediate product was purified by column chromatography using about 300 g of silica gel eluted with a 1:2 mixture of ethyl acetate/hexane.

Collections containing the desired product were combined. Solvents were removed under vacuum to give about 15 g of intermediate (89% yield). The intermediate was a light yellow oil.

### EXAMPLE I(d)

Anhydrous tetrahydrofuran (200 ml) and hexamethylphosphoramide (46 ml) were added to a 1L 3-necked RB flask equipped with a mechanical stirrer, an additional funnel and a thermometer under N₂ in/outlet. The mixture was cooled to −70° C. 3-methyl-butyne (18.1 ml) was added and a clear solution was produced. 2.5M n-BuLi in hexane (90.7 ml) was added dropwise at −70° C. for about 25 minutes. The solution was gradually warmed to −30° C. and stirred for 1 hour at about −25° C. to about 40° C. 5-Bromovaleric acid (16 g in 40 ml of anhydrous THF) was added dropwise at −30° C. for about 10 minutes. The solution was gradually warmed to room temperature and stirred overnight under N₂. A TLC of the reaction mixture revealed that the reaction was completed.

The reaction mixture was cooled to about 10° C. and acidified with 3N HCl (80 ml) to a pH of about 3. Water (200 ml) was added. The solution was extracted with ethyl acetate (2×300 ml). The organic layers were combined, washed with brine (200 ml), dried over anhydrous Na₂SO₄ and filtered. Solvents were removed under vacuum at about 35° C. to give a light yellow oil (wt.=18.7 g). The product was purified by column chromatography with silica gel (approx. 350 g) and eluted with a 1:2 mixture of ethyl acetate/hexane.

Collections containing the desired product were combined. Solvents were removed under vacuum to give a pale yellow oil (wt.=14.5 g) after drying under vacuum overnight.

### EXAMPLE I(e)

Tetrahydrofuran (240 ml, inhibited with BHT) and hexamethylphosphoramide (60 ml) were added to a 1L 3-necked RB flask equipped with a mechanical stirrer, addition funnel and thermometer under nitrogen blanket. This mixture was cooled to −70° C. 3-Methyl-1-butyne (15.6 g) was then added followed by dropwise addition of n-BuLi (92 ml) at an internal temperature of less than −50° C. The mixture was then gradually warmed to −30° C. over a period of 1 hr. A solution of 5-bromovaleric acid (20.7 g) in tetrahydrofuran (40 ml) was the added while maintaining the internal temperature below −30° C. The reaction mixture was warmed up to room temperature gradually and stirred overnight. A TLC of the reaction mixture at this stage showed consumption of all of the bromovaleric acid and the reaction was worked up.

The reaction mixture was cooled to 5-10° C. and then 3M HCl (100 ml) and water (150 ml) were added so that the internal temperature did not rise above 15° C. The solution was then extracted with ethyl acetate (2×200 ml) and the organic layer dried over sodium sulfate and concentrated under vacuum to give a crude step 1 intermediate product. The crude intermediate product was purified by flash chromatography using 10:1 silica gel to the substrate and eluted with a 1:2 mixture of ethyl acetate/hexane. Product fractions were combined and concentrated to provide the step 1 intermediate product (8-methyl-6-nonynoic acid) which was then dried under vacuum to constant weight. The intermediate product produced was a light yellow oil. The amount of intermediate produced weighed 15 g (yield=89%). The spectral data for the step 1 intermediate product was as follows: ¹H NMR (CDCl₃) δ 2.56-2.48 (m, 1H), 2.38 (t, 2H), 2.18 (dt, 2H), 1.75 (br q, 2H), 1.53 (br q, 2H), 1.13 (d, 6H); MS 167 (M⁻−1); GC: 100%.

### EXAMPLE I(f)

Anhydrous THF (5 L, inhibited with 250 ppm BHT) and HMPA (1.3 L) to a 22 L 4-necked round bottom flask equipped with mechanical stirrer, thermometer, additional funnel, and argon in/outlet. The resulting mixture was cooled to −60° C., and 3-methyl-1-butyne (509 mL) was added. n-BuLi (2.5M, 502 mL) was then added at temperature no greater than −60° C. The reaction mixture was gradually warmed to temperature no greater than −30° C. and maintained for about one hour. A solution of 5-bromovaleric acid (450 g) in anhydrous THF (900 mL, containing 250 ppm BHT) was added through an additional funnel at temperature no greater than −30° C. The reaction mixture was gradually warmed to room temperature and stirred overnight. The reaction was monitored by TLC.

The reaction mixture was cooled with an ice bath to ≦10° C., and quenched with water (10 L) portionwise at ≦30° C. The aqueous layer was acidified to a pH of about 2-3 using cold 6N HCl and extracted with ethyl acetate twice (10L, 6 L). The ethyl acetate extracts were combined and washed with brine (10L). After drying over anhydrous Na₂SO₄, the ethyl acetate solution is filtered and concentrated to dryness under vacuum at about 45° C. to give a crude product.

The crude product was purified using normal phase Biotage chromatography using ethyl acetate/hexanes (1:2) as eluents. Collections containing the desired product were combined and solvents removed under vacuum to produce 237 g (77% yield) of the product as a pale yellow oil.

### EXAMPLE I(g)

8-Methyl-6-nonynoic acid (1.4 g) was dissolved in MTBE (10 ml). The solution was basified to pH 10-11 and extracted with MTBE twice (2×8 ml). The aqueous layer was acidified to a pH of about 2-3 using cold 6N HCl and extracted with MTBE (2×8 ml). The MTBE extracts were combined, washed with brine (4 ml). The organic layer was dried over anhydrous Na₂SO₄, filtered, and concentrated to dryness under vacuum at to give the product.

### Step 2: Reduction of 8-methyl-6-nonynoic Acid

The second step in the synthesis of trans capsaicin involves the synthesis of a second intermediate (8-methyl-6-nonenoic acid) by reduction of 8-methyl-6-nonynoic acid. 8-methyl-6-nonynoic acid was successfully reduced in the laboratory by Examples II(a)-(g) as follows:

### EXAMPLE II(a)

The 8-methyl-6-nonynoic acid was dissolved in anhydrous tetrahydrofuran (30 ml) and t-BuOH (0.42 ml) in a 250 ml 3-necked RB flask equipped with a thermometer, a magnetic stirrer and a condenser under nitrogen in/outlets. The solution was cooled to −40° C. Ammonia was condensed to the flask at about −40° C. to about −50° C. Sodium was added piece wise. Addition of the sodium pieces turned the solution to a dark blue color. The solution was stirred for about 30 minutes at about −40° C. to about −45° C. NH₄Cl (1.7 g) was added (approximately 32 mmol) and the mixture was gradually warmed to room temperature. The ammonia evaporated overnight. A TLC of the reaction mixture revealed a yield of from about 30% to about 40% of product.

The reaction mixture was worked-up. Cold water (100 ml) was added and the temperature was increased from about 18° C. to about 24° C. The solution was then acidified with 6N HCl to a pH of about 3. The solution was extracted with ethyl acetate (2×80 ml). The ethyl acetate layers were combined, washed with brine, dried over anhydrous Na₂SO₄ and filtered. The solvents were removed under vacuum. A light yellow oil intermediate product was produced.

### EXAMPLE II(b)

8-Methyl-6-nonynoic acid, anhydrous tetrahydrofuran and t-BuOH were added to a 3-necked RB flask equipped with a magnetic stirrer, an acetone-dry ice condenser and a thermometer under nitrogen in/outlet. The solution was cooled to about −50° C. Ammonia was condensed to the reaction flask at −40° C. Sodium slices were added piece wise for about 10 minutes. The solution was warmed to

−33° C. and stirred for about 2 hours. A TLC of the reaction mixture revealed a yield of from about 30% to about 40% of product. Additional sodium (approx. 0.3 g) was added. NH₃ gradually evaporated. The reaction mixture was warmed to room temperature and stirred overnight.

NH₄Cl (1 g) was added and the solution was stirred for about 30 minutes. The solution was then quenched with water and extracted with ethyl acetate (2×60 ml). The ethyl acetate extracts were combined, washed with brine, dried over anhydrous Na₂SO₄ and filtered to remove solvents under vacuum. A light yellow oil intermediate product was produced.

### EXAMPLE II(c)

8-Methyl-6-nonynoic acid was added to a 500 ml 3-necked RB flask equipped with a mechanic stirrer, a condenser, and a thermometer under nitrogen in/outlet. The solution was cooled to about −40° C. Condensed ammonia was added to the flask, the mixture was stirred until a solution was obtained. Sodium was added piece-wise until the blue color of the mixture sustained. NH₄Cl was added and the resulting mixture was stirred overnight to evaporate NH₃.

Water (100 ml) and ethyl acetate (60 ml) was added. The mixture was then acidified with 6N HCl to a pH of about 3. The organic layer was separated and the aqueous layer was extracted with ethyl acetate (60 ml). The ethyl acetate layers were combined, washed with brine and dried over anhydrous Na₂SO₄.

### EXAMPLE II(d)

8-Methyl-6-nonynoic acid (10 g; approx. 10 ml) was diluted with anhydrous tetrahydrofuran (200 ml, inhibitor free) and anhydrous t-BuOH (7 ml). The solution was cooled to about −50° C. Ammonia (approx. 300-400 ml) was condensed to the mixture.

Lithium was added piecewise (3-4 g) over 30 minutes. TLC of the reaction mixture 30 minutes after the addition of lithium revealed that no starting material was present. The reaction mixture was then stirred overnight under Argon for removal NH₃ evaporation. A further TLC revealed that some by-product existed.

Anhydrous tetrahydrofuran (approx. 200 ml) was added followed by addition of NH₄Cl (approx. 30 g). The mixture was stirred for 30 minutes. Ice-water (approx. 400 ml) was added portion-wise into the mixture. The mixture was then extracted with ethyl acetate (3×300 ml). A TLC of the reaction mixture revealed that two extractions were enough.

The first two extractions were combined, washed with brine (200 ml), dried over anhydrous Na₂SO₄ and filtered. Solvents were then removed under vacuum at about 35° C., which produced about 8.7 g of a yellow oil intermediate product.

### EXAMPLE II(e)

8-Methyl-6-nonynoic acid (4 g), anhydrous tetrahydrofuran (120 ml, inhibitor free) and t-BuOH (2.8 ml) were added to a 1L 3-necked RB flask equipped with a condenser, a thermometer and a magnetic stirrer under Argon in/outlet. The mixture was cooled to about −55° C. to about −45° C. NH₃ (approx. 150-200 ml) was condensed to the flask. Lithium (0.1-0.15 g/ea.) was added piece wise at −60° C. to about −45° C. and the mixture was stirred until a dark blue color disappeared before adding the next piece of lithium. A TLC after addition of 0.4 g lithium revealed approximately 40% to about 50% conversion. Additional lithium (0.75 g) was then added and the completion was observed by TLC. The reaction mixture was stirred for an additional hour (temperature −45° C. to −42° C.). NH₄Cl (2 g) was added portion-wise at approximately −45° C. to about −42° C. The mixture was gradually warmed to room temperature overnight. A TLC of the reaction mixture revealed a clean product.

The reaction mixture was cooled to about 5° C. and quenched with ice-water (200 ml). The temperature increased to about 25° C. The mixture was then acidified with 6N HCl (approx. 50 ml added portion wise at 25° C.) to a pH of about 2 to about 3. The mixture was then extracted with ethyl acetate (2×300 ml). The ethyl acetate layers were combined, washed with brine (200 ml) dried over anhydrous Na₂SO₄ and filtered. The dried mixture was concentrated under vacuum at about 30° C. to give a light yellow oil intermediate product (wt.=3.8 g)

The intermediate product was purified by flash column chromatography using silica gel (approx. 100 to about 110 g) eluted with a 1:3 mixture of ethyl acetate/hexane.

Collections containing the desired product were combined. Solvents were removed under vacuum to give a pale yellow intermediate product (wt.=3.7 g).

### EXAMPLE II(f)

8-Methyl-6-nonynoic acid (4 g), anhydrous tetrahydrofuran (120 ml, inhibitor free) and t-BuOH (2.8 ml) were added to a 1L 3-necked RB flask equipped with a condenser, a thermometer and a magnetic stirrer under Argon in/outlet. The mixture was cooled to about −55° C. to about −45° C. NH₃ (approx. 150-200 ml) was condensed to the flask. Lithium (approx. 4-5 g) was added at a temperature from about −65° C. to about −50° C. for about 1 hour and 40 minutes. The resulting mixture was then stirred at −35° C. for about 30 minutes and monitored by TLC until disappearance of the starting material was observed.

The solution was quenched with NH₄Cl (approx. 20 g) portionwise until blue color faded. The mixture was gradually warmed to room temperature and stirred overnight.

Ice-water (approx. 400 ml) was added portion-wise and the resulting mixture was acidified with 6N HCl (approx. 150 ml) to a pH of about 2 to about 3. The mixture was extracted with ethyl acetate (2×400 ml). The extracts were washed with brine (300 ml), dried over anhydrous Na₂SO₄, filtered and concentrated under vacuum to give a pale yellow oil (wt.=14.2 g). The crude product was purified by column chromatography using silica gel (approx. 300-350 g) eluted with a 1:2 mixture of ethyl acetate/hexane.

Collections were combined and solvents were removed under vacuum and the product was dried under vacuum to produce 12.2 g (86% yield) of intermediate product.

### EXAMPLE II(g)

Tetrahydrofuran (200 ml, inhibitor free), acid (14 g) and t-butanol (7.7 g) were added to a 1L 3-necked RB flask equipped with a mechanical stirrer, additional funnel and thermometer under Argon. The resulting mixture was cooled to −50° C. with dry ice/acetone bath. Ammonia (approx. 200 ml) was condensed to the reaction mixture. Lithium was then added portion-wise to the flask until a blue color persists. The reaction mixture was stirred below −35° C. and monitored by TLC until complete conversion of the starting material observed. The reaction was quenched by adding 20 g of ammonium chloride at −40° C. Gas chromatography confirmed synthesis of the trans (E) isomer only.

After warming to room temperature and evaporation of the ammonia over a period of 10 hrs, ice-water (400 ml) was added and the mixture was then acidified to a pH of 2-3 with 6N HCl. The mixture was then extracted with ethyl acetate (2×400 ml). The combined organics were washed with brine (300 ml) and dried over sodium sulfate (100 g), filtered and concentrated under vacuum to yield second intermediate product. The second intermediate product was a pale yellow oil which was purified by flash chromatography on silica column to yield 12.2 g of yellow oil (86% yield) of pure trans 8-methyl-6-nonenoic acid. The spectral data for the second intermediate product was as follows: ¹H NMR (CDCl₃) δ 5.43-5.29 (m, 2H), 2.35 (t, 2H), 2.22 (m, 1H), 2.02 (m, 2H), 1.65 (m, 2H), 1.41 (m, 2H), 0.95 (d, 6H). MS 169 (M⁻−1).

### EXAMPLE II(h)

8-Methyl-6-nonynoic acid (225 g), anhydrous tetrahydrofuran (2.7 L, inhibitor free) and t-BuOH (154 ml) were added under argon to a 22 L 4-necked RB flask equipped with a condenser, a thermometer and a mechanical stirrer. The mixture was cooled to about −55° C. to about −45° C. Ammonia (approx. 4 L) was condensed to the flask. Lithium (approx. 30-35 g) was added portionwise at a temperature no greater than −33° C. and stirred for no less than 30 minutes while maintaining a dark blue color. NH₄Cl (143 g) was added portionwise. The resulting mixture was gradually warmed to room temperature and the ammonia evaporated.

Ice-water (3 L) was added portionwise. The resulting mixture was acidified using 6N HCl to pH≦3 and extracted with ethyl acetate (2×4.5 L). The organic layers were combined, washed with brine (5 L), dried over sodium sulfate, and filtered. Solvents were removed under vacuum to give a crude product.

The crude product was purified by Biotage column chromatography (ethyl acetate/hexane 1:3) to give 214 g (94%) of product as a pale yellow oil. Gas chromatography confirmed a trans/cis (E/Z) ratio of about 94:3 to about 94:5.

### Step 3: Activation of 8-methyl-6-nonenoic Acid

The third step in the synthesis of trans capsaicin involves the synthesis of a third intermediate (an acid halide) by activation of 8-methyl-6-nonenoic acid. 8-Methyl-6-nonenoic acid was successfully converted to acid chloride in the laboratory by Examples III(a)-(c) as follows:

### EXAMPLE III(a)

8-Methyl-6-nonenoic acid was added to a 50 ml 3-necked RB flask equipped with a condenser, an additional funnel, a thermometer and a magnetic stirrer. Thionyl chloride (SOCl₂) 3.9 ml was added dropwise at room temperature for about 25 minutes. The solution was then heated with a water bath at about 50° C. to about 60° C. for about 1 hour. Excess thionyl chloride was removed under vacuum at about 40° C. to about 45° C. to yield about 3 g of a third intermediate product (acid chloride; a light brownish yellow oil).

### EXAMPLE III(b)

8-Methyl-6-nonenoic acid (12 g) was added to a 100 ml 3-necked RB flask equipped with a magnetic stirrer, an additional funnel, a condenser and a thermometer. Thionyl chloride (15.5 ml) was added dropwise at room temperature under nitrogen for 30 minutes. The solution was then heated for about 1 hour to bring the temperature form about 50° C. to about 65° C. or about 74° C. which produce a brown solution. The excess thionyl chloride was removed under vacuum at about 40° C. to about 42° C. to give about 13.4 g of a brown oil. The intermediate product was then dried under vacuum at 40° C. for 1 hour.

### EXAMPLE III(c)

8-Methyl-6-nonenoic acid (12 g) was added to a 100 ml 3-necked RB flask equipped with a magnetic stirrer, additional funnel, condenser and thermometer. Thionyl chloride (25.2 g) was then added dropwise over a period of 30 minutes. The reaction mixture was then heated to 65-74° C. over a period of 1 hr. Excess thionyl chloride was removed under vacuum and crude acid chloride product was produced. The crude acid chloride product was then used in a fourth synthesis step without further purification to produce trans capsaicin.

### EXAMPLE III(d)

8-Methyl-6-nonenoic acid (200 g) was treated with thionyl chloride (419 g). The reaction mixture was then heated at about 70° C. for about 1 hr. Excess thionyl chloride was removed under vacuum to produce 249 g of a crude product. The crude acid chloride was then used in a fourth synthesis step without further purification.

### Step 4: Coupling of Benzylamine Derivative to the Acid Halide

The fourth step in the synthesis of trans capsaicin involves the synthesis of the trans capsaicin end product by coupling of a benzylamine derivative to the acid halide. The benzylamine derivative was successfully coupled to the acid halide in the laboratory by Examples III(a)-(c) as follows:

### EXAMPLE IV(a)

4-Hydroxy-3-methoxy benzylamine HCl salt (3.35 g) and dimethylformamide (10 ml) were added to a 100 ml 3-necked RB flask equipped with an additional funnel, a thermometer and a magnetic stirrer under nitrogen. 5N NaOH (7 ml) was added portion-wise at room temperature. The mixture was stirred at 35° C. for 30 minutes. The mixture was then cooled to about 0° C. to about 5° C. Acid chloride (produced in step 3) in anhydrous ether (30 ml) was added dropwise at about 0° C. to about 5° C. or 10° C. for about 20 minutes. An additional 5 ml of anhydrous dimethylformamide was added. The mixture was gradually warmed to room temperature and stirred under nitrogen overnight.

Water (150 ml) was added. The mixture was extracted with ethyl acetate (1×100 ml and 1×50 ml). The ethyl acetate extract was washed with 1N HCl (2×60 ml) followed by saturated NaHCO₃ (2×100 ml) and brine. The extract was then dried over anhydrous Na₂SO₄ and filtered. Solvents were removed under vacuum at about 35° C. to about 40° C. to give a thick light orange/pink residue (wt.=3.4 g).

The crude product was purified by column chromatography using from about 150 to about 160 g of silica gel eluted with a 1:1 mixture of ethyl acetate/hexane.

Collections from the column purification were combined, concentrated under vacuum at about 40° C. and dried under vacuum to produce 3.1 g of the desired product as a white solid.

### EXAMPLE IV(b)

4-Hydroxy-3-methoxy benzylamine hydrochloride (13.4 g) and dimethylformamide (40 ml) were added under nitrogen to a 500 ml 3-necked RB flask equipped with a mechanical stirrer, an additional funnel and a thermometer. The mixture was cooled to about 10° C. 5N NaOH (28 ml) was added portion wise with an ice-water bath. The solution was stirred at about 20° C. for 30 minutes then cooled to 5° C. Acid chloride in anhydrous ether (120 ml) was added dropwise at 5° C. for about 1 hour (temperature increased to about 7° C.). The solution was gradually warmed to room temperature and stirred overnight.

Water (400 ml) was added and the resulting mixture then extracted with ethyl acetate (1×400 ml and 2×200 ml). A TLC of the extraction revealed two ethyl acetate extractions were enough. The ethyl acetate extracts were washed with 1N HCl (2×200 ml and 1×100 ml). The organic layers were then washed with NaHCO₃ (2×200 ml and 200 ml), brine, dried over anhydrous Na₂SO₄ and filtered. Solvents were removed under vacuum and the residue co-evaporated with ether (×2) to produce about 21 g of crude product as a light brown sticky residue. The residue was dried overnight under vacuum (wt.=20.2 g).

The product was purified by column chromatography using about 600 g of silica gel eluted with: i) a 2:3 mixture of ethyl acetate/hexane (2L); ii) a 1:1 mixture of ethyl acetate/hexane (3L) and iii) a 3:2 mixture of ethyl acetate/hexane (3L) to obtain a crude compound.

The collections were combined and solvents were removed under vacuum at about 40° C. The product was co-evaporated with ether (×2) and dried under vacuum overnight to give the desired product as a white solid (wt.=14.1 g, 65% yield). HPLC 96% pure.

Overlap collections were also combined and solvents removed under vacuum to 2.4 g of less pure product. An ester by-product (1.4 g) was also separated and characterized.

In additional embodiments of the present invention, purification of the crude product after addition of NaHCO₃, by treatment with 2N NaOH was tested on a test tube scale. The salt produced was found to be soluble in ethyl acetate. Furthermore, it was found preferable to control the reaction temperature (approx. 5-10° C.) when adding the acid chloride to the reaction mixture.

### EXAMPLE IV (c)

4-Hydroxy-3-methoxybenzylamine hydrochloride (13.4 g) and dimethylformamide (40 ml) were added to a 500 ml 3-necked RB flask equipped with a mechanical stirrer, additional funnel and thermometer under nitrogen. The solution was then cooled to 10° C. and 5N sodium hydroxide 28 ml was added dropwise so that the internal temperature was kept below 20° C. The solution was stirred for 30 min at 20° C. and then cooled to 5° C. Once cooled, a solution of acid chloride (13.3 g) in ether (120 ml) was added over a period of 1 hr while maintaining the internal temperature at 3-7° C. The reaction mixture was then stirred over 12 hrs at 20° C.

The reaction mixture was washed with water (400 ml) and ethyl acetate (2×200 ml). The combined organics were washed with 1N HCl (2×200 ml), saturated sodium bicarbonate (2×200 ml) and saturated sodium chloride (200 ml) and then dried over sodium sulfate. Solvent was removed under vacuum and a pink solid was produced and purified by flash chromatography using silica gel and an ethyl acetate/hexane mixture (2:3 to 3:2). Pure fractions were combined and weighed 14.1 g, corresponding to a yield of 65% (HPLC=96% area % purity).

In certain other embodiments, flash chromatography can preferably be avoided in this fourth step since the crude was processed directly to crystallization, which yielded the product in good purity and recovery.

The trans capsaicin obtained by the methods described above was recrystallized using ether/hexane (1:2, 150 ml) at 40-45° C. The mixture was cooled to room temperature over a period of 2 hrs. The white precipitate was filtered and dried to constant weight (weight=12.7 g (Yield=91%)). The spectral data for the trans capsaicin product was as follows: ¹H NMR (CDCl₃) δ 6.86-6.75 (m, 3H), 5.82 (br s, 2H), 5.35-5.32 (m, 2H), 4.34-4.32 (d, 2H), 3.86 (s, 3H), 2.22-2.17 (m, 3H), 2.00-1.95 (m, 2H), 1.75-1.65 (m, 2H), 1.45-1.35 (m, 2H), 0.95 (d, 6H); MS=306 (M+1). Anal. Cald. For C₁₈H₂₇NO₃; C, 70.79; H, 8.91; N, 4.59; Found: C, 70.94, H, 8.94; N, 4.75.

The HPLC Purity (area %) of the synthesized trans capsaicin was 98%.

### EXAMPLE IV(d)

4-Hydroxy-3-methoxybenzylamine hydrochloride (245 g) and dimethylformamide (700 ml) were added to a 12 L 4-necked RB flask equipped with a mechanical stirrer, additional funnel and thermometer under argon. The suspension was then cooled to 10° C. and 5N sodium hydroxide (494 ml) was added dropwise at temperature below 20° C. The solution was cooled to about 10° C. and stirred for 30 min and then cooled to about 5° C. A solution of acid chloride (249 g) in ether (1.7 L) was added dropwise at about 5° C. The reaction mixture was gradually warmed to room temperature and stirred overnight.

The reaction mixture was partitioned between 1N HCl (5L) and ethyl acetate (5L). The aqueous layer was separated and extracted with ethyl acetate (5L). The organic layers were combined, washed with 1N HCl (5L), saturated sodium bicarbonate (3×5L) and brine (5L) and then dried over sodium sulfate and filtered. Solvent was removed under vacuum to produce a crude product. The crude product was purified by two recrystallization from ether/hexane (1:2) to give 237 g (66% yield) of the desired product. HPLC confirmed a ratio of trans/cis (E/Z) of about 98:0.9

### Purification of Capsaicin by Re-Crystallization

### EXAMPLE V(a)

Crude capsaicin (1 g; HPLC 91%) was dissolved in a 1:2 mixture of ether/hexane (15 ml). The mixture was heated to about 40° C. to about 42° C. to dissolve the solid. The mixture was gradually cooled to room temperature while stirring. Off-white particles were produced. The suspension was stirred at room temperature for about 2 hours. The solids were filtered to give off-white solids (wt.=0.74 g).

### EXAMPLE V(b)

Capsaicin (14 g) was dissolved in a 1:2 mixture of ether/hexane (150 ml) at about 40° C. to about 45° C. The solution was gradually cooled to room temperature. A white precipitate formed. The mixture was then stirred at room temperature for about 2 hours. The suspension was filtered, washed with a 1:2 mixture of ether/hexane and dried under vacuum at room temperature for about 2 hours to afford a white solid (wt.=12.7 g; 91% recovery).

### EXAMPLE V(c)

Ether/hexane (1:2, 2.5 L) was added to crude capsaicin (337 g). The resulting mixture was cooled to about 10° C. and stirred for about 2 hours. The solids were filtered, washed with ether/hexane (1:2) and recrystallized again from ether/hexane (1:2) in a similar manner to produce 237 g of the desired capsaicin with HPLC purity of 98% and E/Z ratio of 98:0.9.

### Semi-Preparative Purification of Capsaicin

### EXAMPLE VI(a)

In Example VI(a) 10 g of Capsaicin was purified by HPLC. The isocratic conditions were Methanol/Water (57:43) at a flow rate of 10 ml/minute. The column used was a Waters Symmetry Prep C18 (300×9 mm, 7 u), Serial # T22981A 04.

A. The Materials for use in the HPLC Method were as Follows:

1. Apparatus

Gilson HPLC System with UV Detector

| | | |
|---|---|---|
| Gilson UV/Vis Detector | Model# 119 | Serial# 109H7D083 |
| Gilson Dynamic Mixer | Model# 811C | Serial# 369H7T264 |
| Gilson Interface | Model# 506 | Serial# 369J7PA383 |
| Gilson Liquid Handler | Model# 215 | Serial# 259G7272 |
| Gilson Valvemate | Model# 610 | Serial# 339H7A109 |

2. Chemicals

| | |
|---|---|
| Methanol | Lot# 43080313, EM Science, HPLC grade |
| Water | Lot# 43010, EM Science, HPLC grade |
| Acetronile | Lot# 42165226, EM Science, HPLC grade |
| TEA | Lot# CE619, Burdick and Jackson |
| TFA | Lot# X07476 JT Baker |

B. The Experimental Procedure for the HPLC Method was as Follows:
1. Sample Preparation
500 mg of a 95.4% pure capsaicin sample was dissolved with 1 mL of HPLC grade Methanol. The concentration of the sample prepared for purification was approximately 500 mg/mL.
2. HPLC Conditions for Purification

A Symmetry Prep C18, 300×19 mm-7 micron, was employed for the purification with the following:

| | |
|---|---|
| Mobile Phase: | 57% Methanol/43% Water |
| Flow Rate: | 10.0 mL/min |
| Run Time: | 100 minutes |
| Injection Amount: | 400 μL |
| Wavelength: | 281 nm |

3. HPLC Conditions for Analysis

A synergi Hydro RP, 80A, 250×4.6 mm, and 4 micron column was used to perform the purity checks with the following:

| | |
|---|---|
| Mobile phase: | Gradient |
| | A: Water + 0.1% TEA + 0.1% TFA |
| | B: Acetonitrile |

| | | |
|---|---|---|
| Time | % A | % B |
| 0 | 100 | 0 |
| 10 | 52 | 48 |
| 30 | 52 | 48 |
| 40 | 0 | 100 |
| 50 | 0 | 100 |

| | |
|---|---|
| Flow Rate: | 1 ml/min |
| Run Time: | 50 minutes |
| Injection volume: | 10 μl |
| Wavelength: | 281 nm |

C. Results
The semi-prep purification approach was demonstrated to be successful. The final purity of the Capsaicin was 99.86%. Approximately 7.29 g of Capsaicin were recovered. As 1 gram of crude material was utilized for the feasibility study, the actual amount of crude feedstock processed is approximately 9 grams. The overall recovery yield is ca. 81%.

### EXAMPLE VI(b)

In Example VI(b) a reverse phase semi-prep HPLC method to further purify capsaicin was performed.

A. The Materials for use in the HPLC Method were as Follows:

1. HPLC System and Solvents

a. Hitachi HPLC System with UV Detector.

| | | |
|---|---|---|
| Intelligent Pump | Model# L-7100 | Serial# 1158-047 |
| Diode Array Detector | Model# L-7455 | Serial# 1005-030 |
| Auto Sampler | Model# L-7255 | Serial# 1128-005 |
| Interface | D-7000 | Serial# 1127-021 |
| Degasser (ERC) | N/A | Serial# 102899N0880 |

b. HPLC Columns (Waters):

Analytical Column: Symmetry C18, 250×4.6 mm 5μ, Serial# W20801D0 41 Semi-prep Column: Symmetry C18, 300×29 mm 7μ, Serial# T20101N 07

c. HPLC Solvents (EM Science)

| | |
|---|---|
| MeOH: | HPLC grade, Lot# 42213232 |
| H₂O: | HPLC grade, LOT# 42347 |

B. The Experimental Procedure for the HPLC Method was as Follows:

1. HPLC Method Developed for Purification:

Extensive analytical HPLC method developments were performed for the further purification. General HPLC conditions evaluated are summarized as follows:

| | |
|---|---|
| Flow rate: | 1.0 mg/ml |
| Detector: | UV = 281 nm |
| Temperature: | RT |
| Sample conc.: | 2 mg/ml in ACN/H₂O (1:1) |

The HPLC columns and mobile phase used were summarized in the following table:

| Column | Mobile Phase |
|---|---|
| Symmetry C18 (250 × 4.6 mm 5μ) | A = H2O, B = ACN, |
| | MeOH, EtOH, THF |
| Luna C8 (150 × 4.6 mm 5μ) | A = H2O, B = ACN |
| Zorbax CB-CN (250 × 4.6 mm 5μ) | A = H2O, B = ACN |
| Luna C18 (250 × 4.6 mm, 5μ) | A = H2O, B = ACN |
| YMC-ODS-AQ S-5 (250 × 4.6 mm, 5μ) | A = H2O, B = ACN |
| | [symbol] = H2O (0.1% TFA), |
| | B = ACN (0.1% TFA) |
| Polaris C18 (250 × 4.6 mm, 5μ) | A = H2O, B = ACN, MeOH |
| u-Bondapak NH2 (300 × 3.9 mm) | A = H2O, B = ACN |
| Ultrasphere C8 (250 × 4.6 mm, 5μ) | A = H2O, B = ACN |
| Diazem CN-1 (250 × 4.6 mm, 5μ) | A = H2O, B = ACN |
| YMC basic (150 × 4.6 mm, 5μ) | A = H2O, B = ACN |
| Kromasil C18 (250 × 4.6 mm, 5μ) | A = H2O, B = ACN |
| Zorbax SB-C8 (250 × 4.6 mm, 5μ) | A = H2O, B = ACN |
| Pfarma C18A (250 × 4.6 mm, 5μ) | A = H2O, B = ACN |
| Pfarma C18B (150 × 4.6 mm, 5μ) | A = H2O, B = ACN |
| Zorbax Rx-sil (250 × 4.6 mm, 5μ) | A = Hex, B = THF, EtOAc |
| | A = DCM, B = EtOAc, THF |

Among all the methods developed, Symmetry C18 (250×4.6 mm, 5μ) with isocratic 52% MeOH in H₂O was preferred. Retention time of nordihydro capsaicin (the impurity) is 60.1 minutes, while capsaicin elutes at 69.1 minutes. As the semi-prep HPLC column is only available with 30 cm length, the mobile phase of this method was slightly modified to accommodate the change.

2. Purification of Capsaicin having 1.7% Impurities by Semi-Preparative HPLC

a. Sample Preparation

The crude Capsaicin sample having 1.7% impurities was dissolved in ca. 20 mL of HPLC grade MeOH.

b. HPLC Condition

| | |
|---|---|
| Column: | Symmetry C18 (300 × 19 mm, 7μ) |
| Mobile Phase: | A = H₂O; B = MeOH |
| Elution: | Isocratic 57˜58% MeOH |
| Temperature: | RT |
| Flow Rate: | 9.5 ml/min |
| Injection Volume: | 400-450 μl |
| Detector: | 281 nm (UV) |

C. Results

By evaluating chromatogram of the crude capsaicin, it was found nordihydro capsaicin was the major impurity although some trace amount of late eluting impurities were also observed. As nordihydro-capsaicin and capsaicin both elute no earlier than 60 minutes, an overlapping injection approach was made to reduce the purification cycle time to 45 minutes, thus reducing solvent assumption as well.

During the course of purification, fractions were collected and analyzed by analytical HPLC to make sure the purity is above 99.0%. All purified fractions were pooled and dried via rotary evaporation at 55-60° C.

The purification method was demonstrated to be successful. 9.85 grams of capsaicin was processed with final purity greater than 99.9%. The overall recovery of the purification was 80%.

| Compound Name | Capsaicin |
|---|---|
| Weight (g) | 9.58 |
| Purification Recovery | 80% |
| HPLC Purity | >99.9% |
| NMR analysis, Mass | Consist with the structure |
| Loss on Drying (105° C., 4 hours) | 0.1% |

### EXAMPLE VII

### COMPARATIVE EXAMPLE

Initially, a four-step process was proposed for the synthesis of trans-capsaicin, which included: a) formation of a dianion; b) alkylation of the dianion; c) reduction of the dianion; and d) coupling to a benzylamine derivative to obtain trans-capsaicin. The four-step process is as follows:

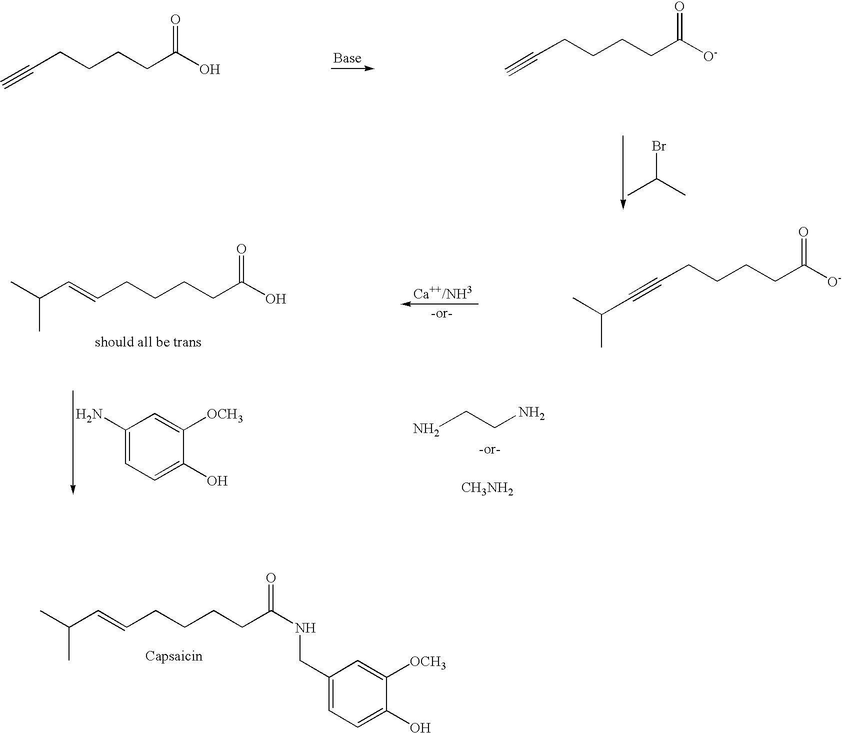

In the first test, alkylation of the 6-heptynoic acid was attempted first using a 2-iodopropane as the alkylating reagent and LDA, NaH and LiNH₂ as the bases to form the alkene anion. Only starting 6-heptynioc acid was recovered. These results suggested that elimination of HI from isopropyl iodide (E2) was exclusive instead of the desired nucleophilic substitution to form the desired isopropyl alkyne.

Alkylation of 6-heptynoic acid was further tested using 2-bromopropane and isopropyl mesylate as the alkylating reagent and NaH and n-BuLi/AlCl₃ as the bases. No desired product was observed under these conditions.

In further alkylation tests, dianion formation was tested. In particular, 6-heptynoic acid (2 g) was treated with LDA (3 equiv.) under conditions similar to those used for the previous isopropyl halide reactions. The reaction mixture was then treated with benzyl bromide. Product formation was confirmed.

In yet another approach, orthoester protection of the carboxylic acid group of 6-heptynoic acid followed by alkylation of the alkyne function was tested. Alkylation with benzyl bromide was tested as a model reaction using 300 mg of the orthoester of 6-heptynoic acid using n-BuLi and ethyl magnesium chloride as a base, respectively.

The use of n-BuLi base produced product/starting compound in a ratio of 7:2. Use of ethyl magnesium chloride produced a small amount of product having a product to starting material ratio of 1:2.

In another test, Grignard, Samarium-Mediated alkylation was tested by alkylation of the orthoester with isopropyl iodide on a 300 mg scale using samarium iodide-samarium (SmI₂) and ethyl magnesium bromide, respectively. No product formation was observed from either reaction.

In another test, trans-8-methyl-6-nonenoic acid was synthesized through a Wittig reaction followed by isomerization. Crude capsaicin was (1.4 g thick oil, approx. 84% trans and 12% cis isomer) was purified by crystallization from ether/hexane (1:3) to give 0.4 g of an off-white solid. 91.5% purity was shown by HPLC with a trans/cis ratio of 91.5/6.7. The cis isomer appeared less crystalline and a mixture (0.3 g) was obtained from sticky residue.

In another test, alkylation of the orthoester of 6-heptynoic acid with isopropyl bromide under Grignard conditions was tested, but no desired product was produced.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1. A method for preparing trans-capsaicin, comprising:
a) alkylating 3-methyl butyne with halovaleric acid or to obtain 8-methyl-6-nonynoic acid;
b) reducing said 8-methyl-6-nonynoic acid to obtain trans-8-methyl-6-nonenoic acid;
c) activating said 8-methyl-6-nonenoic acid to obtain an acid halide or activated acid derivatives; and
d) acylating 4-hydroxy-3-methoxybenzylamine hydrochloride with said acid halide to obtain trans-capsaicin.

2. The method of claim 1, wherein step a) comprises alkylating 3-methyl butyne with ω-haloalkanic acid to obtain ω-alkynoic acid analogues.

3. The method of claim 1, wherein step a) comprises the steps of:
i) mixing anhydrous tetrahydrofuran with hexamethylphosphoramide and cooling said mixture to about −78° C. to about −60° C.;
ii) adding to said mixture of step i) 3-methyl butyne followed by a dropwise addition of a base at a temperature from about −78° C. to about −65° C. to obtain a second mixture;
iii) warming said second mixture up to about −30° C. while stirring; and
iv) adding dropwise a halovaleric acid in anhydrous tetrahydrofuran at a temperature of about −30° C., said halovaleric acid added in a sufficient amount to convert said 3-methyl butyne to said 8-methyl-6-nonynoic acid, then gradually warming to room temperature and stirring to obtain a reaction mixture.

4. The method of claim 2, further comprising:
i) adding hydrochloric acid to said reaction mixture and extracting said reaction mixture with ethyl acetate; and
ii) washing said extracted reaction mixture with brine to yield a crude product.

5. The method of claim 3, further comprising:
i) purifying said crude product; and
ii) removing solvents under vacuum to provide a step a) intermediate product.

6. The method of claim 5, wherein said crude product is purified by column chromatography.

7. The method of claim 5, wherein said crude product is purified by acid-base extraction.

8. The method of claim 5, wherein said crude product is purified by vacuum distillation.

9. The method of claim 5, wherein said step a) intermediate product is 8-methyl-6-nonynoic acid.

10. The method of claim 3, wherein said halovaleric acid is selected from the group consisting of bromovaleric acid, chlorovaleric acid, fluorovaleric acid, iodovaleric acid and astatinovaleric acid, 1-mesyloxyvaleric acid, 1-tosyloxyvaleric acid.

11. The method of claim 10, wherein said halovaleric acid is bromovaleric acid.

12. The method of claim 3, wherein 1,2-dimethyl-3,4,5,6-tetrahydro-(1H) pyrimidinone is substituted for hexamethylphosphoramide in step i).

13. The method of claim 4, wherein said base is selected form the group consisting of n-BuLi, sec-BuLi, t-BuLi, lithium di(isopropyl) amide, sodium hydride, sodium amide, lithium amide, methyl lithium, methyl magnesium bromide, ethyl magnesium bromide, alkyl or aryl magnesium halides or mixture thereof.

14. The method of claim 13, wherein said base is n-butyllithium.

15. The method of claim 1, wherein step b) comprises the steps of:
i) dissolving said 8-methyl-6-nonynoic acid in a mixture of anhydrous tetrahydrofuran and t-butyl alcohol to obtain a solution and cooling said solution to about −55° C. to about −40° C.;
ii) condensing ammonia to said solution to a temperature of about −50° C. to about −33° C.;
iii) adding sodium piece-wise and stirring at a temperature from about −45° C. to about −30° C. and stirring for a sufficient period of time to dissolve said sodium, and
iv) adding ammonium chloride, warming to room temperature an allowing the ammonia to evaporate to obtain a reaction mixture.

16. The method of claim 15, wherein additional lithium is added after step iii).

17. The method of claim 15, wherein step iii) comprises adding lithium at a temperature from about −65° C. to about −45° C. and stirring for a sufficient period of time to dissolve said lithium.

18. The method of claim 15, further comprising:
i) adding water to said reaction mixture;
ii) acidifying said reaction mixture with hydrochloric acid to a pH of about 2 to about 3;
iii) extracting said reaction mixture with ethyl acetate, washing with brine and drying over anhydrous sodium sulfate; and
iv) filtering and removing solvents under vacuum to obtain a step b) intermediate product.

19. The method of claim 18, wherein said step b) intermediate product is trans-8-methyl-nonenoic acid.

20. The method of claim 17, wherein step ii) is omitted.

21. The method of claim 15, wherein lower alkyl amines are substituted for said ammonium of step ii).

22. The method of claim 15, wherein sodium is substituted for said lithium of step iii).

23. The method of claim 15, wherein secondary butyl alcohol (sec-BuOH), ethyl alcohol (EtOH), or other alkyl alcohols are substituted for said t-butyl alcohol of step i).

24. The method of claim 15, wherein lithium and liquid ammonia or sodium and liquid ammonia are substituted for said lithium, said tetrahydrofuran and said liquid ammonia.

25. The method of claim 17, further comprising the steps of:
i) stirring said reaction mixture overnight to evaporate said ammonia;
ii) adding additional anhydrous tetrahydrofuran and ammonium chloride, stirring said mixture for a sufficient time to neutralize excess lithium;
iii) adding ice-water portionwise;
iv) extracting said mixture with ethyl acetate, washing with brine and drying over anhydrous sodium sulfate; and
v) filtering and removing solvents under vacuum to produce a step b) intermediate product.

26. The method of claim 17, further comprising the steps of:
i) cooling the reaction mixture and quenching with ice-water;
ii) acidifying said mixture with hydrochloric acid added portion-wise to a pH of about 2 to about 3;
iii) extracting said mixture with ethyl acetate, washing with brine and drying over anhydrous sodium sulfate;
iv) filtering and concentrating under vacuum at a temperature of about 30° C. to obtain a crude product.

27. The method of claim 26, further comprising the step of purifying said product by flash column chromatography to obtain a step b) intermediate product.

28. The method of claim 26, further comprising the step of purifying said crude product by vacuum distillation.

29. The method of claim 1, wherein step c) comprises the steps of:
i) adding dropwise a thionyl halide to said 8-methyl-6-nonenoic acid at room temperature to form a solution;
ii) heating said solution at about 50° C. to about 75° C. for a sufficient period of time to convert said 8-methyl-6-nonenoic acid to said acid halide; and
iii) removing excess thionyl halide under vacuum to obtain a step c) intermediate product.

30. The method of claim 29, wherein said thionyl halide is thionyl bromide.

31. The method of claim 29, wherein said thionyl halide is thionyl chloride.

32. The method of claim 29, wherein said step c) intermediate product is an acid halide.

33. The method of claim 32, wherein said acid halide is acid bromide.

34. The method of claim 32, wherein said acid halide is acid chloride.

35. The method of claim 32, wherein said acid halide is an activated carboxylic acid.

36. The method of claim 35, wherein said activated carboxylic acid is an imidazolide.

37. The method of claim 35, wherein said activated carboxylic acid is an carbodiimide.

38. The method of claim 1, wherein step d) comprises the steps of:
i) mixing 4-hydroxy-3-methoxy benzylamine hydrochloride and dimethylformamide;
ii) adding portion-wise at room temperature to said mixture of step i) aqueous sodium hydroxide and stirring to obtain a reaction mixture;
iii) adding acid halide in anhydrous ether at a temperature of about 0° C. to about 10° C. for a sufficient period of time to convert said acid halide to an amide; and thereafter
iv) gradually warming said mixture to room temperature and stirring.

39. The method of claim 38, further comprising the steps of:
i) adding water to said mixture and extracting said mixture with ethyl acetate to obtain an ethyl acetate extract;
ii) washing said extract with hydrochloric acid and, thereafter, washing with sodium bicarbonate;
iii) washing said solution with brine and drying over anhydrous sodium sulfate;
iv) filtering and removing solvents under vacuum to obtain a crude trans capsaicin product.

40. The method of claim 39, further comprising the steps of:
i) purifying said crude product by column chromatography to obtain trans-capsaicin product.

41. The method of claim 38, wherein potassium hydroxide, lithium hydroxide, sodium carbonate, potassium carbonate, or an alkyl amine is substituted for said aqueous sodium hydroxide of step ii).

42. The method of claim 38, wherein 4-hydroxy-3-methoxy benzylamine is substituted for said 4-hydroxy-3-methoxy benzylamine hydrochloride of step i).

43. The method of claim 41, wherein said alkyl amine is selected from the group consisting of triethylamine, Hunig's base, 4-dimethylaminopyridine and pyridine.

44. The method of claim 38, wherein tetrahydrofuran, 2-dimethoxyethane, acetonitrile, dichloromethane, chloroform, or methyl ethyl ketone is substituted for said dimethylformamide in step i).

45. A method of purifying the trans-capsaicin product of claim 35, comprising the steps of:
i) dissolving said crude trans-capsaicin product in a mixture of ether/hexane and heating said mixture to about 40° C. to about 45° C.;
ii) cooling said mixture to room temperature or bellow room temperature; and
iii) filtering said mixture to provide a purified trans-capsaicin product.

46. The method of claim 45, wherein step iii) comprises filtering said mixture and washing said mixture with a mixture of ether/hexane and drying under vacuum to obtain a purified trans-capsaicin product.

47. The method of claim 1, further comprising purifying said trans-capsaicin using a semi-preparative HPLC.

48. The method of claim 39, further comprising purifying said crude trans-capsaicin product using a semi-preparative HPLC.

49. The method of claim 40, further comprising purifying said trans-capsaicin product using a semi-preparative HPLC.

50. The method of claim 47, wherein the purification using the semi-preparative HPLC provides for a resulting ultra-purified trans-capsaicin having a purity of about 97% or greater capsaicin.

51. The method of claim 47, wherein the purification using the semi-preparative HPLC provides for a resulting ultra-purified trans-capsaicin having a purity of about 98% or greater capsaicin.

52. The method of claim 47, wherein the purification using the semi-preparative HPLC provides for a resulting ultra-purified trans-capsaicin having a purity of about 99% or greater capsaicin.

53. The trans-capsaicin product produced by the method of claim 47.

54. A capsaicin composition for relieving pain at a site in a human or animal in need thereof consisting essentially of pure trans capsaicin.

55. The composition of claim 54, wherein said trans capsaicin is used for the treatment of nociceptive pain, neuropathic pain, pain from nerve injury, pain from neuralgia, pain from myalgias, pain associated with painful trigger points, pain from tumors in soft tissues, pain associated with neurotransmitter-dysregulation syndromes and pain associated with orthopedic disorders.

56. The composition of claim 54, wherein said trans capsaicin is used for the treatment of orthopedic disorders selected from the group consisting of conditions of the foot, knee, hip, spine, shoulders, elbow, hand, head and neck.

57. The composition of claim 54, wherein said pure trans capsaicin is provided in an injectable formulation.

58. A trans-capsaicin compound comprising about 97% or greater trans-capsaicin.

59. A trans-capsaicin compound comprising about 98% or greater trans-capsaicin.

60. A trans-capsaicin compound comprising about 99% or greater trans-capsaicin.

61. A pharmaceutical composition comprising an ultra-purified trans-capsaicin compound comprising about 97% or greater trans-capsaicin, about 98% or greater trans-capsaicin, or about 99% or greater trans-capsaicin and a vehicle suitable for infiltration or injection.

62. The pharmaceutical composition of claim 61, wherein said vehicle comprises about 20% PEG 300, about 10 mM histidine and about 5% sucrose in water for injection.
